(19) United States
(12) Patent Application Publication
(10) Pub. No.: US 2005/0064590 A1
(43) Pub. Date: Mar. 24, 2005

(54) Cryoconservation on woven textiles

(76) Inventors: Heiko Zimmermann, Saarbrucken (DE); Jorg-Uwe Meyer, St. Ingbert (DE); Gunter R. Fuhr, Berlin (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/502,869

(22) PCT Filed: Jan. 29, 2003
(86) PCT No.: PCT/EP03/00904

(30) Foreign Application Priority Data
Jan. 30, 2002 (DE) 102 03 644.6

(51) Int. Cl. C12N 5/00

(52) U.S. Cl. 435/374; 435/1.1

(57) ABSTRACT

A cryosubstrate, in particular for cryopreservation of biological specimens, having at least one solid-phase element is described. This solid-phase element is equipped for adherent fixation of specimens, with the solid-phase element comprising at least one carrier filament. Furthermore, a method of cryopreservation of biological specimens using such a cryosubstrate is described.

This invention relates to devices for cryostorage of biological specimens, in particular cryosubstrates for biological specimens, and methods for cryopreservation of biological specimens using such devices.

It is generally known that biological specimens may be converted to a frozen state (cryopreservation) for storage and/or processing. For macroscopic specimens such as blood or tissue, numerous techniques have been developed for storing specimens at low temperatures. In modern medicine, genetic engineering and biology, there is a trend toward subjecting smaller and smaller samples and specimens to cryopreservation. For example, small suspension volumes (ml or less) containing suspended cells or cell groups may be frozen.

Cryopreservation of cells from in vitro cultures is performed mainly with the cells in suspension. However, most biomedically relevant cells require contact with a substrate for their replication and proper development. Therefore, specimens are bound to a substrate when frozen, optionally after culturing. An important requirement is for the specimens to be available in an essentially unchanged state after cryopreservation. So far only a few cryosubstrates suitable for low-temperature treatment of cells are known in cryopreservation. These are usually planar systems resembling the in vitro culture surfaces. For example EP 804 073 discloses the order deposition of suspension droplets on planar cryosubstrates. Although planar cryosubstrates have advantages with respect to handling and identification of the cryospecimens, they take up a relatively large amount of space. In addition, transferring specimens to traditional cryosubstrates frequently requires a marked intervention into the ambient conditions, e.g., in the transfer of a suspension to a cryosubstrate. This may be a disadvantage in terms of the properties of the specimen.

Document DE 37 51 519 T2 describes the culturing of stroma cells in vitro on a two-dimensional mesh and then shaping the mesh with the cells growing on it to form a three-dimensional culture carrier for culturing other types of cells such as liver cells. The culture carrier is formed by adhesion and enclosure of the mesh by the stroma cells. DE 37 51 519 T2 also describes storing the culture carriers for culturing the other types of cells in a frozen state. The mesh described in DE 37 51 519 T2 has only limited suitability as a carrier for cryopreservation of biological materials because the culture carrier has a high mesh density with small mesh sizes and as a fixed composite of the mesh and cells, it forms a new functional unit for subsequent cell culturing. The small mesh sizes result in a low mechanical flexibility of the culture carrier. The cells that have grown on the mesh and become interlaced with it cannot be separated from it without being destroyed. In addition, it has been found that cells become detached from the mesh during the freezing process.

Document U.S. Pat. No. 3,997,396 describes the culturing of cells on hollow fibers at room temperature, these hollow fibers being arranged in a reactor in the manner of an in vitro culture surface. To promote culturing, oxygen flows through the hollow fibers. From a practical standpoint, the hollow fibers are not suitable as carriers for cryopreservation of biological materials.

The object of the present invention is to provide improved cryosubstrates, in particular for cryopreservation of biological specimens so that the disadvantages of traditional cryosubstrates can be overcome. These cryosubstrates should permit in particular good utilization of space in cryostorage, a physiological transfer of specimens from suspensions (i.e., a transfer that is adapted to the cell systems) and/or good handling of the specimens. The object of this invention is also to provide improved methods for cryopreservation of biological materials.

These objects are achieved with a cryosubstrate and methods having the features according to patent claims 1 or 16. Advantageous embodiments of this invention are derived from the dependent claims.

A first general aspect of this invention consists of providing a cryosubstrate, in particular for cryopreservation of biological specimens, equipped with at least one solid-phase element for adherent fixation of specimens, whereby the solid-phase element is formed by a plurality of carrier filaments which, in contrast with traditional planar cryosubstrates, form a fabric having multiple fabric regions in which the carrier filaments each have different carrier filament densities (number of filaments per unit of area or volume) and/or different surface coatings. This embodiment of the present invention is associated in particular with the advantages of a greater flexibility in use of the cryosubstrate.

Another general aspect of this invention consists of providing a cryosubstrate with at least one solid-phase element which is formed by a single carrier filament or a single carrier filament strand of multiple carrier filaments. This embodiment of the present invention is associated in particular with the advantages of a simplified and gentle loading of the cryosubstrate which is free of stress for the cells, for example, and permits a rapid freezing process.

A third general aspect of this invention consists of providing a cryosubstrate which has at least one solid-phase element consisting of a plurality of carrier filaments which form a fabric or a carrier filament strand having a carrier filament density such that an average distance (e.g., mesh spacing) between the carrier filaments is greater than or equal to the thickness of the carrier filaments. The distance between the carrier filaments is preferably selected so as to make them permeable for biological cells, i.e., is in the range of 100 μm to 5 mm, for example, in particular 400 μm to 2 mm. This embodiment of the invention may have in particular advantages with respect to the loading of the solid-phase element and the mechanical flexibility of the cryosubstrate. The mechanical flexibility is important in particular in the freezing process and in handling the loaded cryosubstrates.

The first through third general features of this invention mentioned above may be provided according to this invention either individually or in combinations. These measures make it advantageously possible to create a cryosubstrate having a greatly expanded functionality. The cryosubstrate forms a carrier for biological materials as well as a means for supporting the freezing process.

The at least one carrier filament is an elongated, rod-shaped, fibrous or wire-like flexible structure. The inventive solid-phase elements have a number of advantages with respect to loading with specimens, cryopreservation (cooling below the freezing point of water, storage and/or processing) and handling of cryopreserved specimens. A cryosubstrate is in general a carrier for specimens which are subjected to a freezing process and to which the specimens are adherently attached or which surrounds the specimens. Biological specimens such as cells, cell groups, biological tissue or biological tissue fragments can be adherently attached to one or more carrier filaments. To do so, the solid-phase element may be advantageously immersed in at least one suspension with the specimens to be fixed.

Carrier filaments are flexible. Accordingly, depending on the preservation conditions, they may be shaped into a bundle or braid and arranged in a space-saving manner. It is also advantageous that carrier filaments can be severed using simple tools, e.g., cutting devices so that the sampling in a deep frozen or thawed state is facilitated.

According to the first preferred embodiment of this invention, the solid-phase element includes a plurality of carrier filaments which form at least partially a fabric. The term fabric here is understood in general to be a composite produced from many intersecting carrier filaments including a braided strand or fiber or a multidimensional braided, hooked, knit or crocheted material. The design of the solid-phase element as a fabric has the advantage of an increase in stability without any loss of flexibility in loading or any loss of the advantages in cryopreservation.

In addition, a fabric also has an expanded range of applications with respect to culturability of the specimen on the cryosubstrate and modification of the functionality of the cryosubstrate, e.g., by filament coating, forming a composite or integration of additional devices. Two-dimensional or three-dimensional fabric structures in particular have advantages with regard to ease of handling and identifiability, as also obtained with conventional planar cryosubstrates.

An inventive cryosubstrate may be characterized in particular by a fabric-like solid-phase element in which the various fabric areas are bonded together by individual carrier filaments spaced a distance apart. Carrier filaments spaced a distance apart form bridges between fabric regions or fabric segments. The intervals between the carrier filaments are preferably larger than biological single cells, e.g., approximately 100 μm to 2 mm, so that even when loaded, breakthroughs or holes are formed in the cryosubstrate. Through this measure the functionality of the cryosubstrate can be further improved to advantage. If the fabric of carrier filaments used according to this invention has openings or holes, the specimens may be attached to their edges. This further increases the advantage of specimen fixation in a minimal contact area. The solid-phase element may also be designed so that specimens freely suspended between the fabric areas and/or the openings are used in cryopreservation.

And advantageous design of the inventive cryosubstrate is characterized by the formation of the solid-phase element as an elongated, e.g., cylindrical fabric. The fabric forms a one-dimensional structure macroscopically like a fiber and can thus be loaded and preserved with a high flexibility.

As an alternative, the fabric may be designed to be planar or three-dimensional. This design has the special advantage of a high uptake capacity for biological specimens in the adherent or suspended state.

Carrier filaments used according to this invention consist in general of elongated aggregates of organic, inorganic or mixed origin, e.g., textile yarns, metallic wires or the like. Carrier filaments may be coated in at least partial regions according to an advantageous embodiment of this invention in order to promote or suppress the adherent fixation of specimens. This has the advantage of localization of specimens in certain regions of the cryosubstrate.

One particular advantage of the fabric used according to this invention consists of the fact that other functional elements can be integrated into the cryosubstrate. For example, metallic wires as electrodes or optical elements may be woven into the solid phase. Other examples are described below.

A subject of the present invention is thus also to provide methods of cryopreservation of biological specimens using the inventive cryosubstrates. An inventive method is characterized in particular by specimen loading of at least one carrier filament. According to a preferred embodiment of this invention, specimens are inherently attached to one or more carrier filaments, e.g., in the form of a fabric. Alternatively, they may be enclosed by a textile fabric in an initially freely suspended state and deep frozen during cryopreservation.

This invention also yields the following advantageous properties:
planar structured or three-dimensional structured permeable substrate structure,
broad spectrum of materials with adjustable properties (e.g., promoting or hindering cell adhesion, etc.),
reproducible production at a low cost,
sterilizability and easy handling in terms of laboratory technique,
easy washing processes which minimize stress for the cells, in particular for introducing and removing cryoprotective substances,
cell-tissue materials are easily removed selectively and in a stable position when in a frozen state,
easy to reach the cells in a deep frozen state,
automatability of the processes, e.g., working with continuous materials,
implementability of the cryosubstrates as one-dimensional structures (e.g., filaments, strands or fibers), as two-dimensional structures (woven, knit, hooked or crocheted) fabrics or as three-dimensional structures (composites or networks),
high bioavailability and biocompatibility.

Special advantages in cryopreservation include:
easy fractionability (even when deep-frozen),
possibility of adding specimens by weaving filaments in the loaded state,
joining carriers, e.g., by “sewing”,
combining different materials (including alternating),
combination with new materials/combinations (possible in particular because different expansion coefficients are tolerated with a loose weave),
multiple uses of the fabric (including as a liquid storage device for supplying cells), and
cooling function optionally provided for the fabric as a nitrogen storage (e.g., in transport of cryosubstrates).

Further details and advantages of this invention are apparent from the following description of the accompanying drawings, which show:

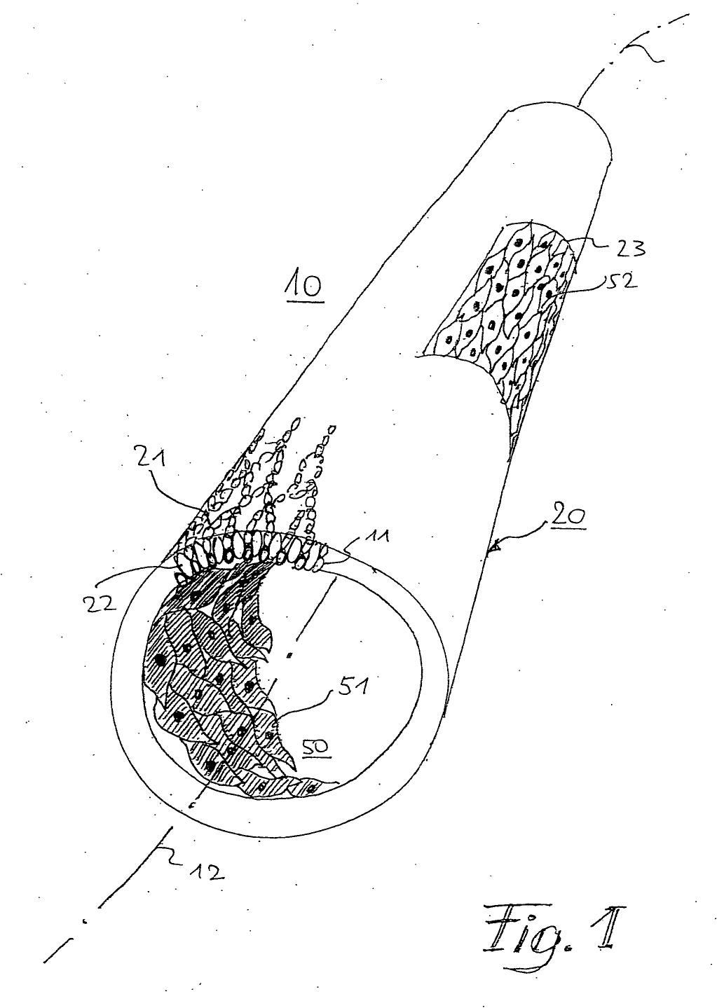

FIG. 1 through FIG. 3 and FIG. 5 through FIG. 10: various embodiments of inventive cryosubstrates, and

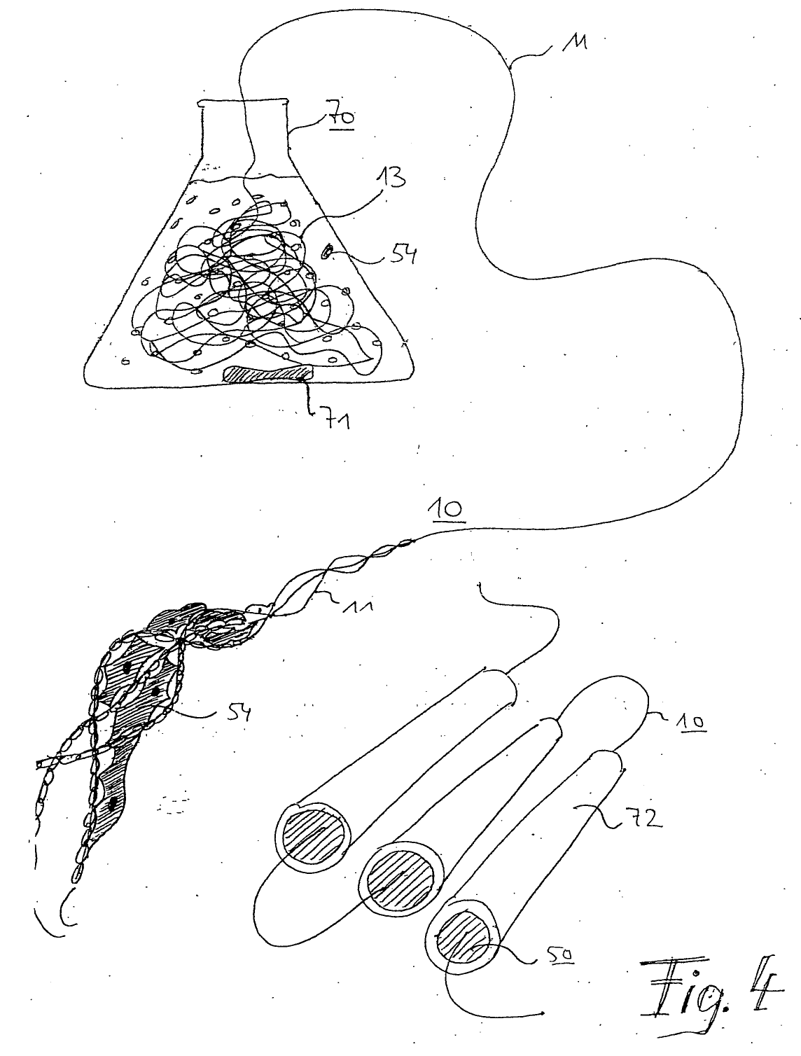

FIG. 4, FIG. 11: illustrations of loading of the inventive cryosubstrates.

Embodiments of inventive cryosubstrates are described below in particular with reference to the design of solid-phase elements of at least one carrier filament. The solid-phase elements shown in the figures may each be used in combination with solid substrate parts as described below. Inventive cryosubstrates are provided for cryopreservation, optionally in combination with culturing.

According to FIG. 1, there is a solid-phase element 10 consisting of a plurality of carrier filaments 11 which are linked together to form a fabric 20 (carrier filament strand). A knitted, crocheted, woven or otherwise created fabric 20 in the form of a long thread or cylindrical hollow body (fabric tubing) is shown as an example. In general, the fabric according to this invention has a homogeneous structure or the structure is divided into individual fabric areas, depending on the application. One possible structural shape of the spun fiber on which this is based (carrier filament 1) is illustrated on the fabric surface 21 and in the sectional view on the forward cross section 22. Biological specimens 50, e.g., cells 51 or cell groups or biological tissue are arranged in the interior of the fabric 20 or on the surface thereof. Reference number 12 denotes the advantageous extensibility of the of the solid-phase element 10.

The fabric 20 may be coated or modified locally in its surface properties (e.g., by molecular layers or a plasma treatment) so that the adhesion of cells is promoted or hindered (or even prevented). For example, a fabric area 23 with carrier filaments treated at the surface is either formed in such a way as to promote adherent fixation of specimens (e.g., cells 53).

A special advantage of the surface modification is obtained in the loading of inventive cryosubstrates. If the system is completely or partially placed in a culture solution being known as such (see FIG. 4) for animal or plant cells, the cells grow in suitable locations and reproduce only in those locations.

The fibrous fabric structure consists, for example, of a textile yarn (thickness in the range of 20 nm to 5 mm, for example). Alternatively, a biocompatible and cold-resistant plastic material (fibers), a composite material or so-called nanofibers or fibers composed of so-called nanotubes based on carbon may also be used. According to an advantageous embodiment of this invention, additional functional elements may also be integrated into the fabric in these embodiments as well as others. For example, metallic filaments may be woven, knotted or otherwise integrated (e.g., stapled or glued) into the fabric 20.

The metallic filaments may, for example, have the functions of measurement electrodes, locally acting heating elements, stimulators, actuators or sensors. The sensors may be for example PT100 temperature sensors that are integrated into the fabric 20. The electrodes and/or stimulators may be used to test for vitality, to control the release of active ingredients or for identification. In addition, metallic filaments may also be provided for forming intended separation points in the fabric. Furthermore, it is possible to integrate optical waveguides such as glass fibers and optional elements into the fabric, in particular for analytical or control purposes.

Another advantage of the inventive use of carrier filaments consists of their ability to be interwoven according to known methods from textile technology. Carrier filaments, optionally with the function elements, can be manufactured on machines that are essentially already available.

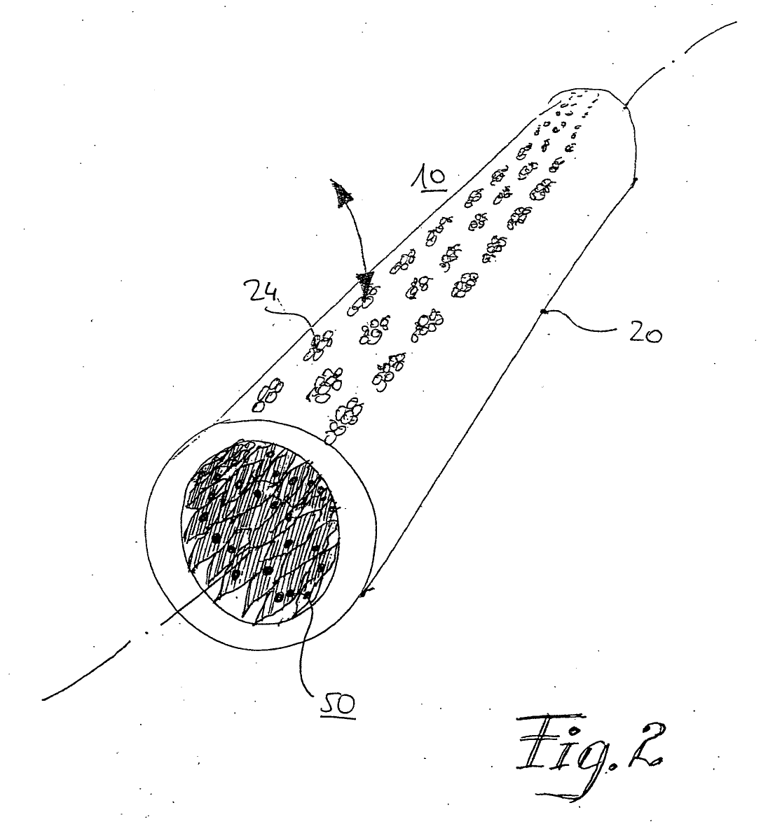

FIG. 2 shows another cylindrical fabric 20 in the interior of which are cells 53 (adherent or suspended in a solution). The fabric is densely woven and/or treated at the surface in various ways. There are fabric areas 24 having a high permeability (see arrow), e.g., for cells for migrating in and out or only for a nutrient solution. Alternatively or additionally, modifications of the filaments may be provided in the fabric areas 24, promoting or hindering adherent attachment for cells, for example. These modifications include, for example, coatings of fibronectin, collagen, poly-L-lysine. This heterogeneous structure of the solid-phase element is advantageous for cell reproduction, washing out solutions and removing the cells before and after freezing.

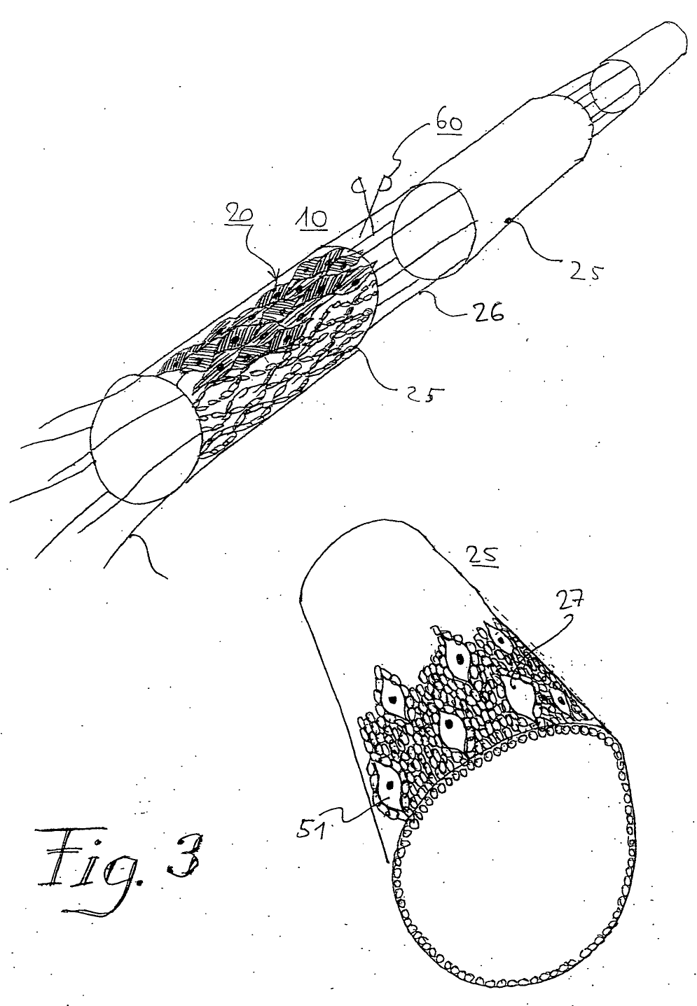

FIG. 3 shows a solid-phase element 10 in the form of a fabric thread 20 with alternating denser and less dense fabric regions 25, 26 which can be separated if necessary. The less dense woven regions 26 consist for example of individual carrier filaments or fabric with a lower filament density. The regions 26 form predetermined separation points which can be severed with a cutting device 60 (e.g., scissors). Tearing or breaking at the intended separation points are also possible for removing individual fabric areas from the solid-phase element 10 and from the cryosubstrate. Such a separated section 25 is shown at the bottom of FIG. 3.

Section 25 is woven in a heterogeneous manner and is treated so that the fabric has open areas or “holes” 27 in the fabric. These holes 27 are preferably of such dimensions that even one or more cells 51 can fit into them or settle on them. Heterogeneous cell systems can be created and cryopreserved in this way. Inventive cryosubstrates thus advantageously form a prerequisite for effective working or designing of biological tissue (so-called tissue engineering).

FIG. 4 illustrates a method of loading the inventive cryosubstrates with biological specimens. The biological specimen is in a fluid in a culture vessel 70. A stirrer device 71 may be arranged in the culture vessel 70 to create a movement in the specimen liquid (optionally with a liquid culture medium). The cryosubstrate includes as a solid-phase element in the example shown here a woven, knit or otherwise structured fiber (consisting here of individual hooked fibers or individual carrier filaments 11). The solid-phase element 10 is inserted into the vessel 7 for the suspension culture with adherently growing cells. The solid-phase element 10 forms a coil 13, for example, in the culture vessel 7. The cells 44 will colonize the coil 13, grow on it and multiply there. For cryopreservation or for further use, the colonized fiber is pulled out periodically in pieces or continuously (optionally through additional solutions) and frozen in suspension or with a liquid covering it. The freezing is performed using refrigeration media which are known from traditional methods of cryopreservation, e.g., in the vapor of liquid nitrogen.

As an alternative to substrate loading in the culture vessel 70, according to this invention tubular culturing devices 72 with closed walls may be provided for applying specimens 50 to the solid-phase element 10 so that the solid-phase element 10 is additionally protected and surrounded by a solution. According to the invention, a culturing device 72 may be used as a solid substrate part. Culturing and/or cryopreservation of the specimen may be performed by maintaining the composite of the solid-phase element 10 and the culturing devices 72.

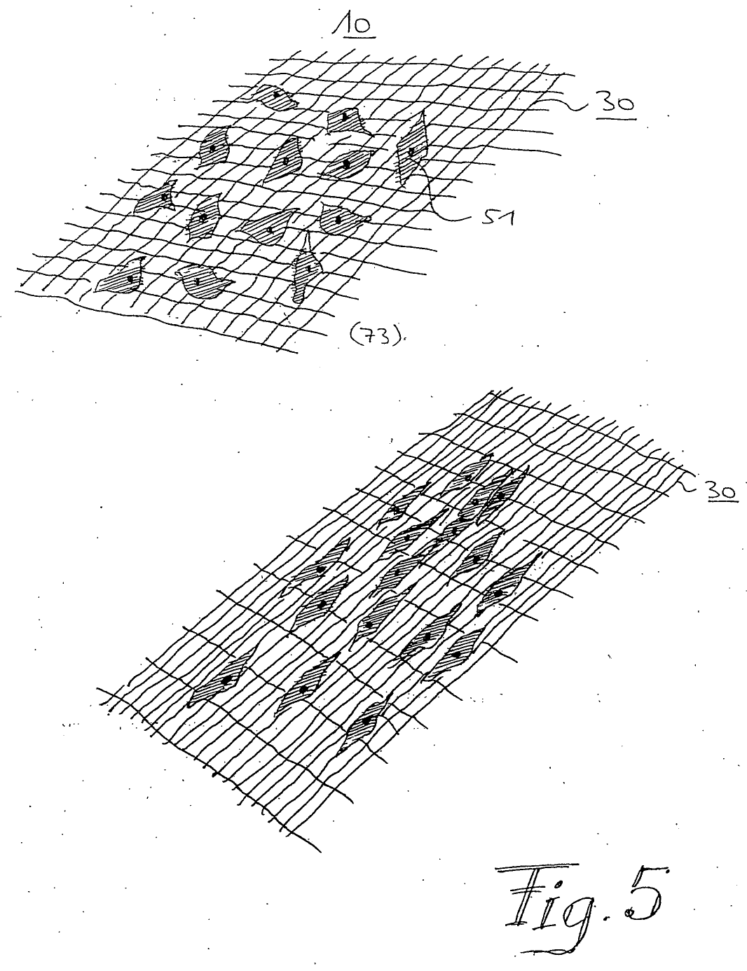

FIG. 5 shows as an example the colonization of a planar fabric area 30 of an inventive cryosubstrate with cells 51. The carrier filament density is so low that the mesh spacing between the carrier filaments is greater than the thickness of the carrier filaments. The fabric area 30 is in a liquid culture medium 73 (not shown). The cells first grow in an irregular random alignment. According to an advantageous embodiment of the invention, the formation of the solid-phase element (here: the fabric 30) may be provided during the adherent fixation of the specimen. Due to a gradual or rapid deformation (e.g., tensile force parallel to the alignment of the fabric threads) the cells have a preferable direction and are further cultured in alignment or frozen. This is illustrated schematically in the lower part of FIG. 5. The deformation of the solid-phase element 10 is also important for the three-dimensional and planar colonization of cryosubstrates for implantation applications. This results in additional advantages for optional following steps in tissue engineering of biological tissue.

According to an advantageous embodiment of this invention, multiple solid-phase elements, each consisting of carrier filament fabrics, may be joined together to form a fabric composite. The connection is preferably accomplished by adapted joining methods such as sewing or interweaving.

FIG. 6 shows as an example a three-dimensional fabric composite 40 which consists of two fabric regions (fabric areas) 31, 32 which are kept at a distance (e.g., 2 μm . . . 100 μm) by a spacer or a spacer fabric 41. The spacer 41 is provided with perforations 42 so that there are hollow spaces in the fabric composite 40 into which the cells 51 can migrate in the suspension. The cells are attached, frozen and thawed in the hollow spaces. Advantages of this fabric composite 40 consist of a good substrate contact, the creation of discrete cell areas and facilitated replacement of the solution.

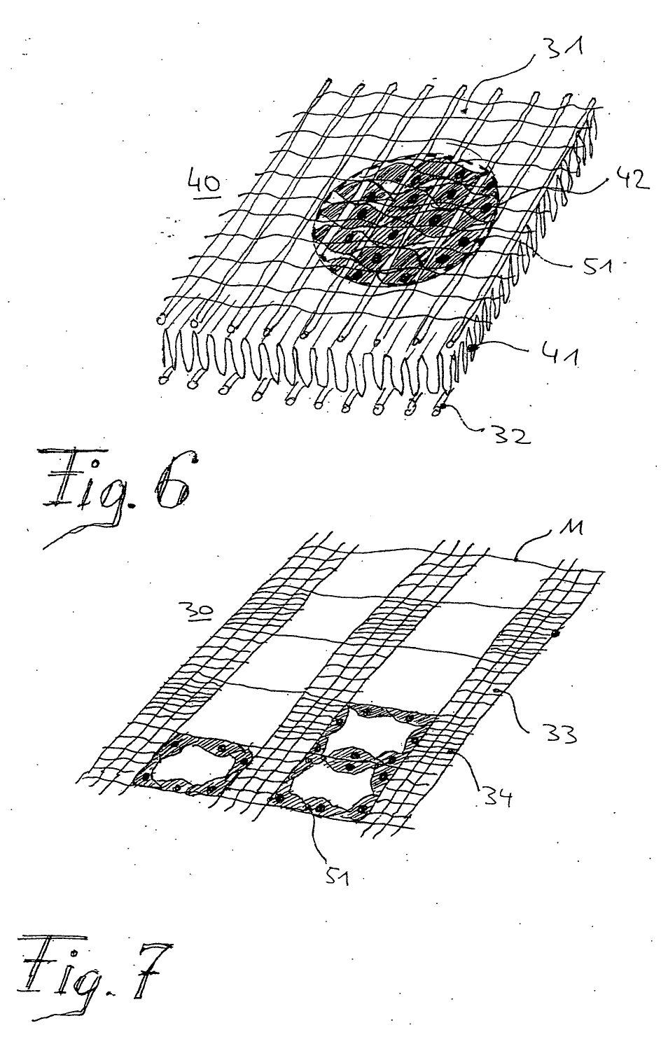

FIG. 7 shows a planar woven substrate 30 consisting of various tightly woven or knit fabric regions 33, 34. The fabric regions 33, 34 are joined together or they are joined by individual connecting fibers or carrier filaments 11. The filaments of the fabric 30 may be treated at the surface so as to promote adherence of the specimen in the case of cells only at the carrier filaments 11 but hindering adherence of cells to the fabric regions 33, 34. Therefore the cells 51 settle preferentially on the carrier filaments 11. Alternatively, converse conditions may be selected in which adherence in the fabric regions 33, 34 occurs preferentially. The cryosubstrate illustrated in FIG. 7 has the special advantages of a selective dissolution of specimens and rapid removability of individual cell strands. These may have the same properties as those already described above.

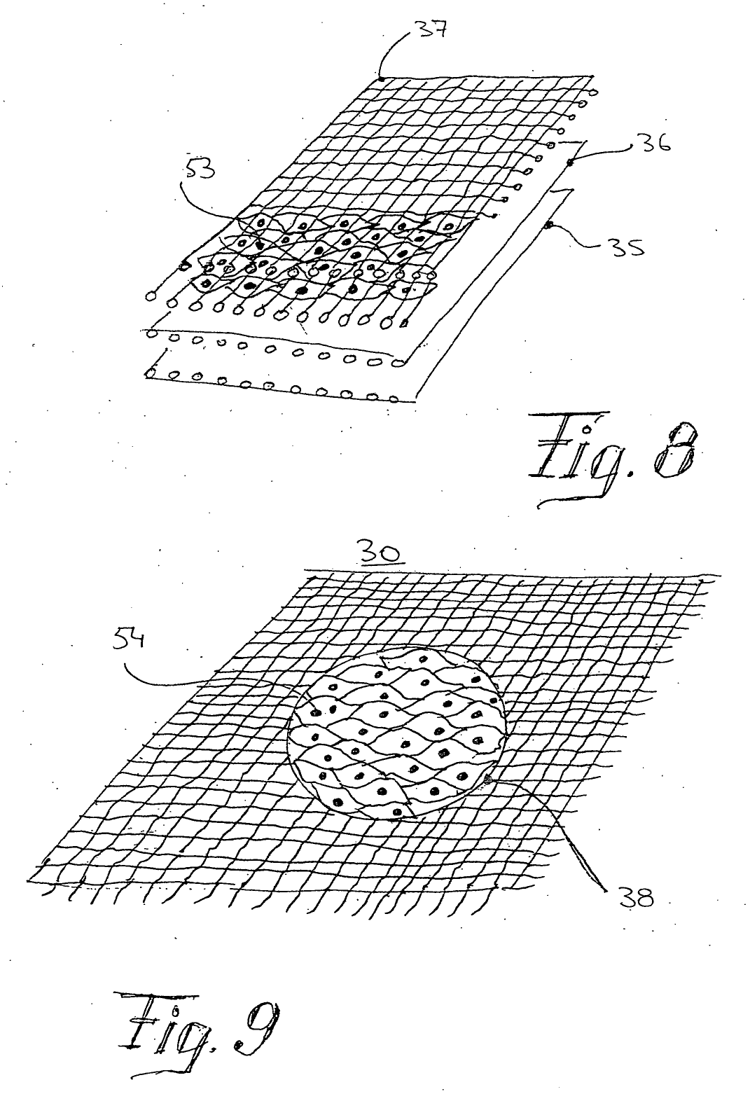

FIG. 8 shows a multilayer structure of two-dimensional fabric regions 35, 36, 37 which are arranged a distance apart (e.g., by weaving) so that one or more cell layers 53 can settle and propagate between them. The advantage of such arrangements consists of the communication among different cell types in different fabric layers which may nevertheless be connected by diffusion in a molecular signal contact or directly via cell-cell bonds. The applications lie mainly in providing cryopreserved specimens for tissue engineering and medical applications, e.g., in trauma surgery and implantation medicine.

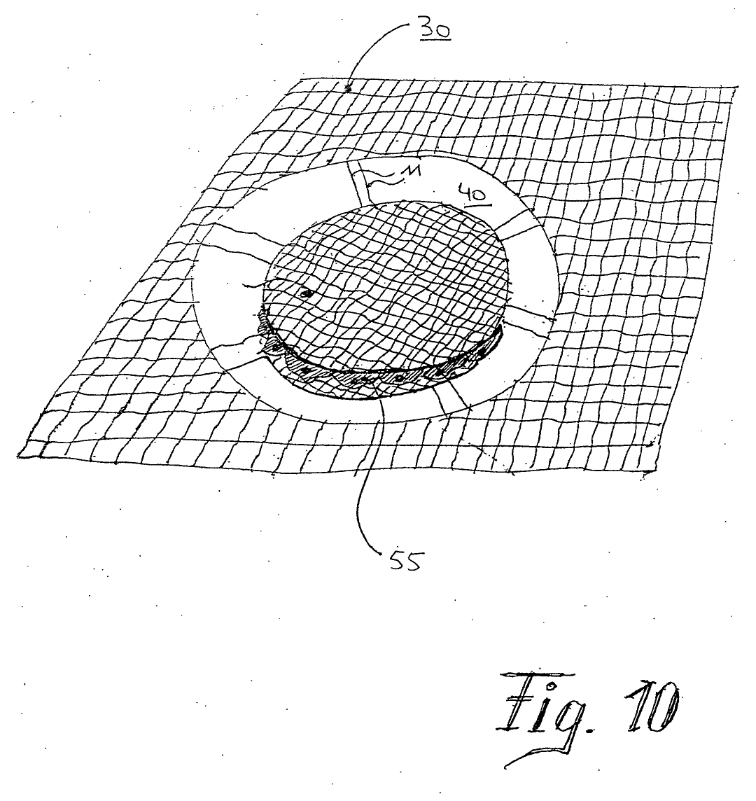

FIG. 9 shows another embodiment of a planar fabric such as that used for example in the embodiment according to FIG. 8. The surface here is structured or coated so that the cells 54 can adhere only in the fabric regions 38. Subsequently the part of the fabric outside of the region 38 remains free of cells. Such structures allow highly variable possibilities for combinations of cell accumulations in direct or indirect contact. The size of the cell area 38 may be miniaturized down to a single cell size.

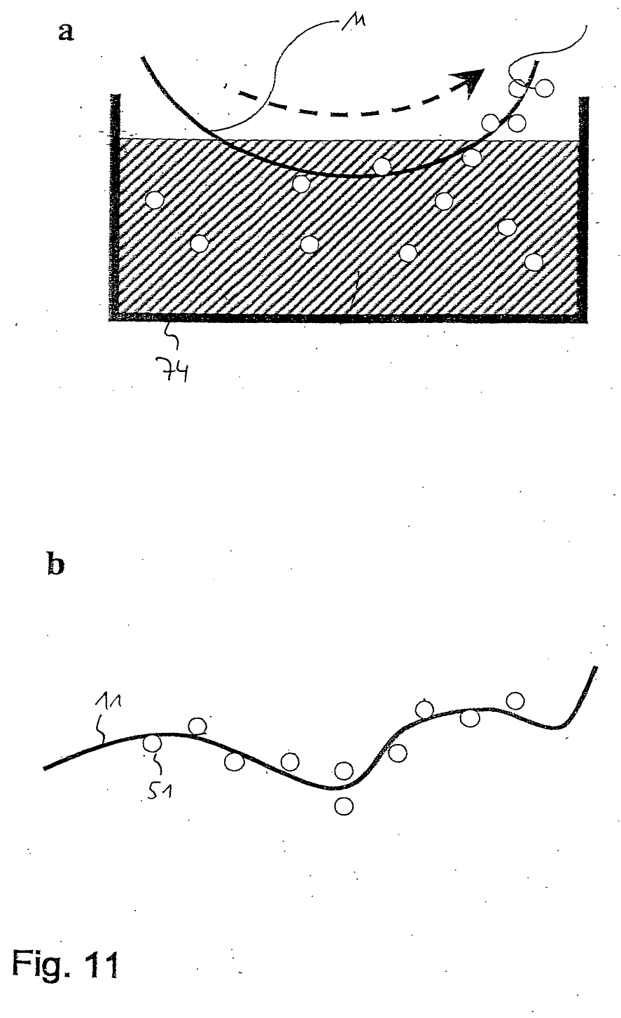

FIG. 10 shows a detail of a fabric 30 having a fabric composite 40 in which cells 55 are enclosed. The fabric composite is connected by a few bonds to the fabric 30. Cells frozen on an area in this way may advantageously be removed (by punching or tearing) in a frozen state in individual stages. The fabric structure according to FIG. 10 can be provided to advantage in the form of materials from individual pieces to continuous webs.

A combination of one or more fabric-like solid-phase elements with a solid substrate carrier may be provided in all embodiments of this invention. The solid substrate carrier consists, for example, of a planar substrate surface on which the solid-phase element is arranged or it consists of a structured body with specimen reservoirs. The specimen reservoirs form compartments in the structured body in which at least one solid-phase element may be arranged and frozen with adherently attached or enclosed specimens, optionally also with a liquid nutrient medium or suspension.

FIG. 11a and FIG. 11b illustrate additional details of the loading of an inventive cryosubstrate or a filament-like specimen body, where the fabric or filament 11 is drawn through a specimen reservoir 74 (e.g., a cell suspension) and thereby loaded with the specimen. FIG. 11b shows the loaded solid-phase element in which the specimen remains on the fabric in the form of small, partially separated specimen volumes or in the form of directly adherent cells.

The features of this invention disclosed in the preceding description, the drawings and the claims may be of relevance either individually or in various combinations for the implementation of this invention in its various embodiments.

1. A cryosubstrate for cryopreservation of biological specimens, said cryosubstrate comprising at least one solid-phase element, wherein said at least one solid-phase element is adapted for adherent fixation of specimens, and comprises:
a plurality of carrier filaments forming at least partially a fabric, and
multiple fabric regions in which the carrier filaments each have at least one of different carrier filament densities and different surface modifications.

2. The cryosubstrate according to claim 1, wherein the fabric regions are connected by individual carrier filaments spaced a distance apart mutually.

3. The cryosubstrate according to claim 1, wherein the fabric has an elongated strand shape or a tubular shape.

4. The cryosubstrate according to claim 3, wherein the fabric is in the form of a cylindrical hollow body in at least some sections.

5. The cryosubstrate according to claims 1, wherein the fabric has a planar or curved shape.

6. The cryosubstrate according to claims 1, wherein the fabric has a three-dimensional shape.

7. The cryosubstrate according to claim 6, wherein the fabric comprises multiple fabric areas arranged one above another.

8. The cryosubstrate according to claim 7, wherein the fabric areas are arranged one above another with a distance between them.

9. The cryosubstrate according to claims 1, wherein the fabric has holes.

10. The cryosubstrate according to claim 9, wherein the holes are of such dimensions that a peripheral edge of the holes permits fixation of specimens of individual cells or cell groups.

11. A cryosubstrate for cryopreservation of biological specimens, said cryosubstrate comprising at least one solid-phase element adapted for adherent fixation of specimens, wherein the at least one solid-phase element is formed by an individual carrier filament or by an individual carrier filament strand of multiple carrier filaments.

12. The cryosubstrate according to claim 11, wherein a fabric at least partially formed by more than one individual carrier filament, or the carrier filament strand has a carrier filament density such that average distances between carrier filaments are greater than or equal to a thickness of the carrier filaments.

13. The cryosubstrate according to claim 11, wherein the individual carrier filament has a modification in at least predetermined partial regions, said modification being such as to promote adherent fixation of specimens.

14. The cryosubstrate according to claim 11, wherein the solid-phase element is arranged on a solid substrate surface or in a substrate compartment.

15. The cryosubstrate according to claim 11, wherein the solid-phase element is equipped with at least one member selected from the group consisting of electrodes, stimulators, active ingredient sources, sensors, identification devices and devices for testing vitality.

16. A method of cryopreservation of biological specimens, said method comprising the steps of:
loading the biological specimens on at least one carrier filament of a cryosubstrate according to claim 1 to provide a loaded cryosubstrate, and
subjecting the loaded cryosubstrate to cryopreservation to cryopreserve the biological specimens.

17. The method according to claim 16, wherein cells or cell groups are adherently attached to the at least one carrier filament and are frozen in a composite with the at least one carrier filament for cryopreservation.

18. The method according to claim 16, wherein the loading of the at least one carrier filament with the biological specimens is accomplished by immersion of the at least one carrier filament in at least one specimen suspension.

19. The method according to claim 16, wherein parts of the cryosubstrate are cut away for sampling.

20. The method according to claim 16, wherein a fabric of carrier filaments is loaded with specimens, whereby the fabric is deformed after adherent fixation of specimens.

21. A method of using fibrous materials as a cryosubstrate for cryopreservation of biological specimens.

22. The cryosubstrate according to claim 1, wherein the fabric or a filament strand of multiple carrier filaments has a carrier filament density such that average distances between carrier filaments are greater than or equal to a thickness of the carrier filaments.

23. The cryosubstrate according to claim 1, wherein the carrier filaments have modifications in at least predetermined partial regions, said modifications being such as to promote adherent fixation of specimens.

24. The cryosubstrate according to claim 1, wherein the solid-phase element is arranged on a solid substrate surface or in a substrate compartment.

25. The cryosubstrate according to claim 1, wherein the solid-phase element is equipped with at least one member selected from the group consisting of electrodes, stimulators, active ingredient sources, sensors, identification devices and devices for testing vitality.

26. A method of cryopreservation of biological specimens, said method comprising the steps of:
loading the biological specimens on at least one carrier filament of a cryosubstrate according to claim 11 to provide a loaded cryosubstrate, and
subjecting the loaded cryosubstrate to cryopreservation to cryopreserve the biological specimens.

27. The method according to claim 26, wherein cells or cell groups are adherently attached to the at least one carrier filament and are frozen in a composite with the at least one carrier filament for cryopreservation.

28. The method according to claim 26, wherein the loading of the at least one carrier filament with the biological specimens is accomplished by immersion of the at least one carrier filament in at least one specimen suspension.

29. The method according to claim 26, wherein parts of the cryosubstrate are cut away for sampling.

30. The method according to claim 26, wherein a fabric of carrier filaments is loaded with specimens, whereby the fabric is deformed after adherent fixation of specimens.
